(19) United States
(12) Patent Application Publication
(10) Pub. No.: US 2006/0155244 A1
(43) Pub. Date: Jul. 13, 2006

(54) Safety catheter device

(76) Inventor: Sergey Popov, Beer-Sheva (IL)

Correspondence Address: Daniel J Swirsky;AlphaPatent Associates, PO Box 2345, Beit Shemesh, 99544, IL

(21) Appl. No.: 10/515,825

(22) PCT Filed: May 22, 2003
(86) PCT No.: PCT/IL03/00421

§ 371 Date: Mar. 13, 2006

(30) Foreign Application Priority Data
May 27, 2002 (IL) 149867

(51) Int. Cl. A61M 5/32

(52) U.S. Cl. 604/162; 604/110

(57) ABSTRACT

A safety catheter device for hemodialysis and donor collection (see FIG. 9) comprises a catheter unit (101), a needle (105) guided by a needle port (114) into the catheter unit to introduce it into patient's vein, an indication channel for blood supply into an indication cavity (109) during catheter emplacement, a blood stream channel for blood supply to hemodialysis or collection set through a blood stream port (112). The minimal cross-sectional area of the blood stream channel is substantially more than one of the indication channel. Forming the blood stream channel is accomplished by retracting the needle and, in version embodiment, by rotating a port unit (111) including the needle and blood stream ports relative to the catheter unit. There are means preventing blood leakage and personnel infecting.

### BACKGROUND OF THE INVENTION

1. Field of the Invention

The present invention relates to medical venipuncture devices for hemodialysis and donor blood collection.

2. Description of the Prior Art

The current venipuncture devices include a catheter unit consisting of a plastic tubular catheter and a catheter hub, as well as a needle unit comprising a needle and a needle hub. The needle has a distal sharp point and an inner passageway, and is housed inside the tubular catheter for introducing this catheter into the patient's vein. After catheter emplacement, blood is collected via the needle's lumen. The needle hub is provided with a coupling means for connection to hemodialysis apparatus or blood collection set.

U.S. Pat. No. 4,160,450 discloses the Outside-The-Needle Catheter Device with a hollow housing. The device includes a tubular catheter with a catheter hub liquid-tightly connected with the hollow housing. There are two versions of the housing—the rigid and the collapsible. The device also includes a needle and a needle hub, which is provided with a distal shank attached to a coupling means. The needle hub is disposed within the hollow housing and its shank extends proximally beyond this housing. In the rigid version of the housing, there is a liquid-tight, slidable connection between the shank and the proximal end of the housing. In the collapsible version of the housing, this connection is immovable. The needle is housed within the catheter and extends beyond its distal end during insertion of the catheter into the vein. The needle is retracted into the housing cavity for device operation, for instance for hemodialysis. The disadvantages of this device are considered below.

The main disadvantage of this device is the low speed of blood flow caused by passing of the all blood flow via narrow internal channel of the needle. This leads to undesirable extending the duration of hemodialysis or blood collection procedure. After inserting the catheter into the vein, it is necessary to completely retract the needle from the catheter into the hollow housing. Therefore, the tightness of the slidable connection between the needle hub shank and the hollow housing in the rigid housing version must be provided throughout the long stroke of the needle retraction. This requires precise details fabrication, which increases the manufacturing cost. In the collapsible housing version, the long flexible housing is awkward to fabricate and assemble.

The speed of blood flow abruptly drops after entering the internal cavity of the hollow housing, increasing the danger of blood clot formation. Moreover, the disturbance of blood laminar flow in the zones of the catheter outlet and the needle inlet can lead to blood cell's injury, i.e. to the impairment of blood quality. After withdrawal of the catheter from the patient's vein, there is a probability of relatively large blood leakage from the device. This relates, to a greater extent, to the collapsible housing version because accidental press onto flexible housing causes abundant blood ejection. As a result, the risk of medical personnel infection arises. The device with the rigid housing contains a locking means in the form of a springy ring located inside the hollow housing cavity. This disposition of the locking means complicates the device's assembly and causes a local vortex motion of the blood, worsening the blood quality. The device has many ergonomics disadvantages. Specifically, it is inconvenient to hold the device with the collapsible housing during transposing the catheter into the extension position. Another disadvantage of the device with the collapsible housing is the presence of air within the housing before a venipuncture. This air can enter the blood stream and cause an embolism.

U.S. Pat. No. 5,370,623 discloses Catheter with Protective Cover and Method of Catheterization. The device includes a needle with a distal sharp point and a needle hub. The device also includes a tubular catheter and a catheter hub. The catheter encircles the needle and has two positions. In the first position, the needle distal sharp point is extended distally beyond the distal catheter end, and in the second position, the catheter is extended distally and covers the needle distal sharp point. There is a manually controlled bayonet-type locking means for mutual fixation of the catheter unit and the needle unit. The transposition of the catheter from the first position into the second position is actuated by a spring mounted between the needle hub and catheter hub, therewith the needle hub serves as a handle. The disadvantages of the device are considered in the following.

The passage of the all blood flow through the narrow internal passageway of the needle significantly decreases the speed of the blood flow, increasing the duration of medical procedure. Moreover, the significant change of the diameter of the blood passageway upon the transition from the needle to the needle shank causes the disturbance of laminar blood flow and leads to the impairment of a blood quality. The fabrication and assembly of the device details are substantially complicated due to the presence of the bayonet type locking means. The lock means is not automatic and, therefore, its actuation entirely depends on the user attention. The likelihood of a blood leakage out of the device arises due to the absence of a sealing means between the catheter and needle hubs. The substantial disadvantage is the use of the needle hub as a handle during transposing the catheter into the extended position by the spring. Since this transposition is very quick and directed to the patient's vein, it can lead to vein injury.

Thus, general disadvantages of the prior art devices are: the increased duration of hemodialysis or blood collection procedure, the impairment of blood quality and the danger of blood clot formation, insufficient safety for patients and medical personnel, the complexity of detail's fabrication and assembly leading to increased manufacturing cost, the blood leakages, and insufficient reliability.

### SUMMARY OF THE INVENTION

The objective of the present invention is the reduction in the duration of hemodialysis and blood collection procedures.

Another objective is the elimination of a blood impairment and blood clot formation during a blood passage through the device.

Another objective is the elimination of patient trauma and medical personnel infection.

Another objective is the elimination of the blood leakage from the apparatus.

Another objective is providing the high reliability of the device operation.

The above noted objectives of the present invention are accomplished with a safety catheter device having: a catheter unit with a tubular catheter and a catheter hub; an indication means having an indication cavity with transparent walls and an indication channel for blood supply from patient's vein into the indication cavity; a penetrating means including a needle with a distal sharp end, needle hub, a needle port located adjacent to said tubular catheter and serving as a passageway for introducing said needle into said tubular catheter, and a penetrating means channel participating in the blood supply into the indication cavity; a port unit including the needle port, as well as a blood stream port for the connection of the tubular catheter with an external device such as a hemodialysis or blood collection set; a blood stream channel including an internal channel of the tubular catheter and the blood stream port and having the minimal area of passage, which is substantially more than one of the indication channel. In the penetrating position of the device, the needle passes through the needle port and the internal channel of the tubular catheter, the needle distal sharp end is extended distally of the tubular catheter distal end, and the indication channel is open for blood supply into the indication cavity. After the introduction of the needle and tubular catheter into the patient's vein, the user retracts the needle by a retracting means into a needle retraction position, wherein the needle is removed from the tubular catheter, the needle distal sharp end is located inside a needle guard and fixed in this position by a stopping means and a locking means. This eliminates the possibility of vein injury and personnel infection with the needle distal sharp end. There is a needle port sealing means between the needle and the needle port, which eliminates the blood leakage through the needle port outside of its limits. The above needle retraction enables the complete opening of the passage of the blood stream channel, which is substantially more than the passage of the indication channel. This allows the substantial increase in the blood rate during the procedure of hemodialysis or blood collection, and, as a result, the reduction in procedure duration.

In version embodiment, the port unit is movable relative to the catheter unit. After the needle retraction, the user displaces the port unit relative to the catheter unit into a blood stream position by a controlling means. In obtained blood stream position, the blood stream port and the tubular catheter are coaxial, forming the blood stream channel with laminar blood flow beyond the needle, thereby eliminating blood cell damages and blood clot formations. There is a unit's sealing means between the catheter unit and the movable port unit, which eliminates the blood leakage during device operation. Moreover, there are a mounting means preventing the catheter and port units from spontaneous disconnection, as well as a fixing means for reliable fixation of the device elements in each of the above positions. After the accomplishment of the medical procedure, the user further displaces the port unit into a finish position, wherein the blood stream port is disconnected from the tubular catheter and sealed to prevent a blood leakage from the device after the withdrawal of the tubular catheter from the patient's vein. Moreover, the distal sharp end is protected from any contact with personnel.

### BRIEF DESCRIPTION OF THE DRAWING

FIGS. 1 to 9 show the safety catheter device with the port unit, which is movable relative to the catheter unit, wherein:

FIG. 1 shows the side view of the safety catheter device in the insertion position.

FIG. 2 shows the top view of the safety catheter device in the insertion position.

FIG. 3 shows the front view of the safety catheter device in the insertion position.

FIGS. 4, 5 show the longitudinal sections of the safety catheter device in the insertion position.

FIGS. 6, 7 show the cross sections of the safety catheter device in the insertion position

FIG. 8 shows the longitudinal section of the safety catheter device in the needle retraction position.

FIG. 9 shows the longitudinal section of the safety catheter device in the blood stream position.

FIGS. 10 to 14 show the safety catheter device with the catheter hub and the device ports made as an integral unit, wherein:

FIG. 10 shows the side view of the safety catheter device in the insertion position.

FIG. 11 shows the top view of the safety catheter device in the insertion position.

FIG. 12 shows the longitudinal section of the safety catheter device in the insertion position.

FIG. 13 shows the cross sections of the safety catheter device in the insertion position

FIG. 14 shows the longitudinal section of the safety catheter device in the blood stream position.

### DETAILED DESCRIPTION OF THE INVENTION

An explanation of the present invention is offered with the references made to the attached drawings.

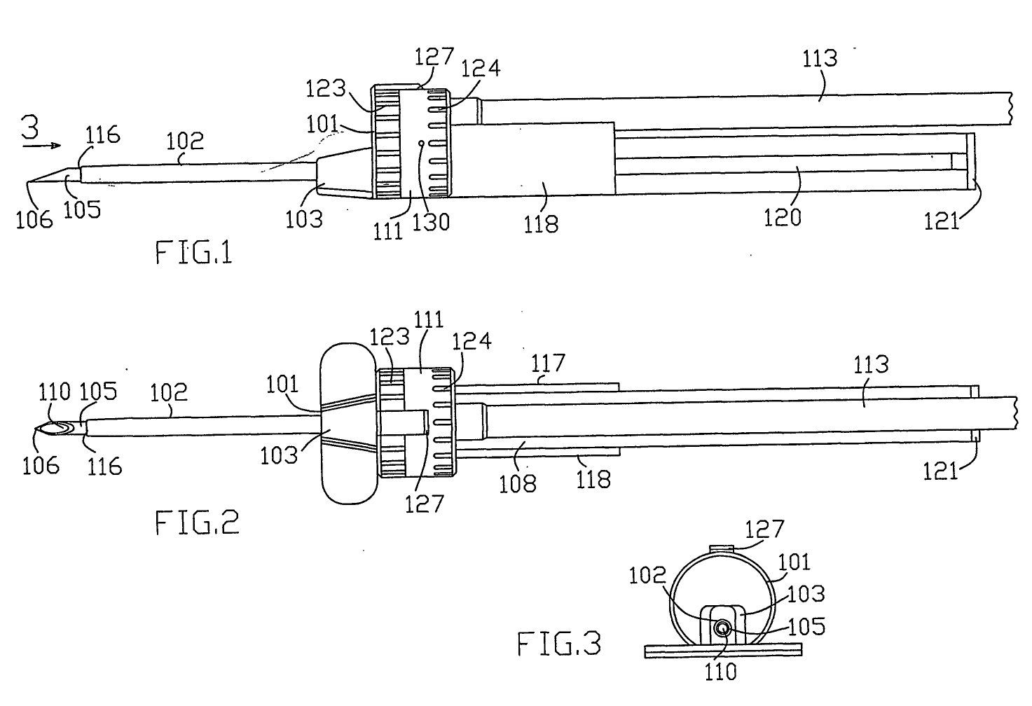

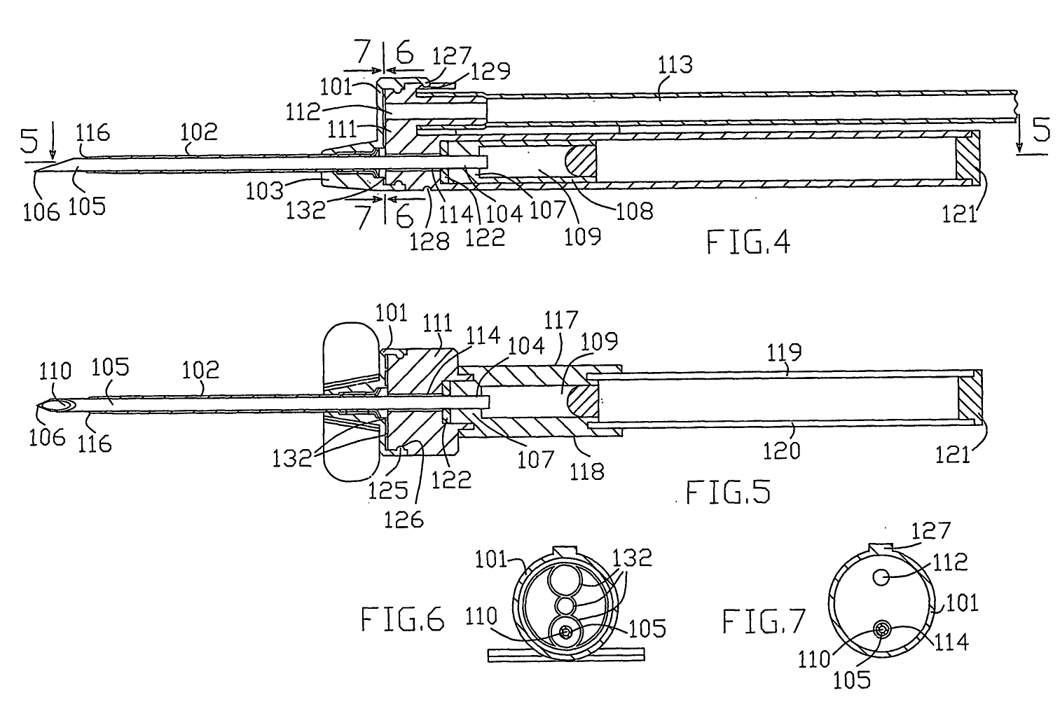

The safety catheter device shown in FIGS. 1 to 9 is comprised of: catheter unit 101 including tubular catheter 102 and catheter hub 103; penetrating means 104 designed for the introduction of the tubular catheter into the patient's vein and including needle 105 with distal sharp end 106 and needle hub 107; the indication means 108 designed for the indication of getting needle distal sharp end 106 into the patient's vein and having an indication cavity in the form of flash chamber 109 attached to needle hub 107, and an indication channel 110 made as an internal channel of needle 105 for blood supply into flash chamber 109; a port unit 111 rotatably connected with catheter unit 101 and having blood stream port 112 for the communication of tubular catheter 102 with the external device (not shown) via tube 113, and needle-port 114 serving as a passageway for introducing needle 105 into tubular catheter 102. Internal channel 115 of tubular catheter 102 and blood stream port 112 are designed to form the blood stream channel for blood flow during hemodialysis or blood collection.

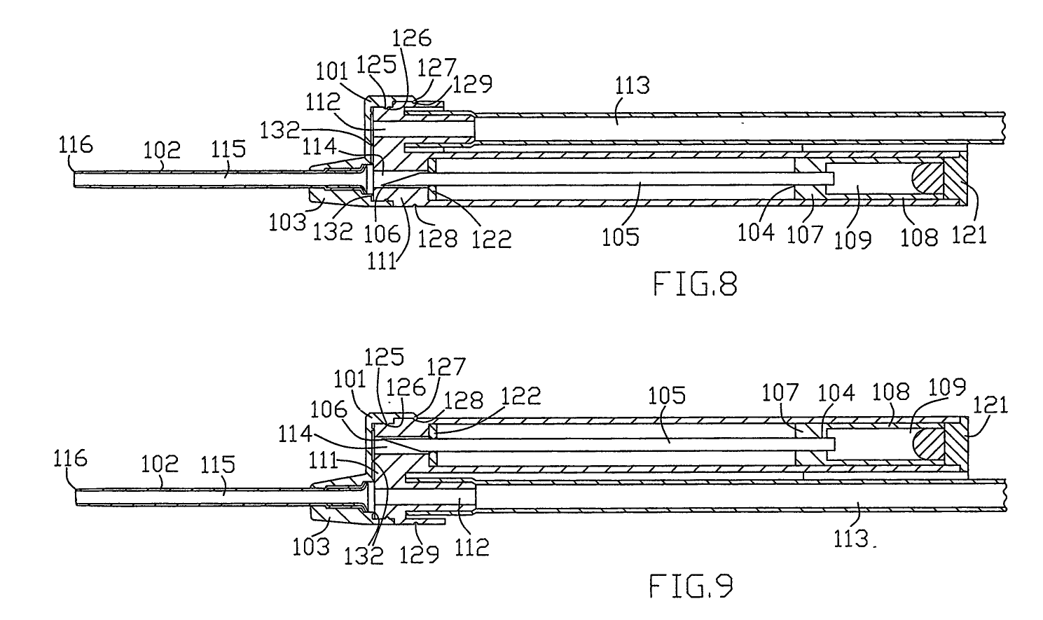

In the penetration position shown in FIGS. 1 to 7, needle 105 is passing through needle port 114 and internal channel 115 of tubular catheter 102 so that needle distal sharp end 106 is extended distally of distal end 116 of tubular catheter 102, and needle internal channel 110 is open for blood supply into flash chamber 109. This position allows inserting needle distal sharp end 106 and tubular catheter 102 into the patient's vein. After the appearance of blood in flash chamber 109, the user manually retracts needle 105 into the needle retraction position shown in FIG. 8. In doing so, he uses the pulling means in the form of external members 117, 118 connected with needle hub 107, accessible and adapted to the operation contact with user's fingers. Lateral, elongated slots 119, 120 serve for the guidance and support of needle hub 107 during the needle retraction. In the needle retraction position, needle 105 is removed from catheter tube 102, needle distal sharp end 106 is located inside needle port 114, which also serves as a needle guard, and member 121 operates as a stopping means preventing needle distal sharp end 106 from the displacement proximally out of guard 114. The device is provided with the needle port sealing means in the form of sealing member 122 preventing the blood leakage through needle port 114 outside of its limits.

The needle retraction creates the possibility for complete opening of the blood stream channel by the communication of tubular catheter 102 and blood stream port 112. For this purpose, the user changes over the device into the blood stream position by the rotation of port unit 111 through 180° relative to catheter unit 101 around the axis, which is parallel to the axis of tubular catheter 102, thereby coaxially communicating internal channel 115 of tubular catheter 102 and blood stream port 112 and forming the blood stream channel (see FIG. 9). In doing so, the user immovably holds catheter unit 101 at its finned surface 123 and rotates port unit 111 holding it at its finned surface 124. During this rotation, the mounting means in the form of circular projection 125 on catheter unit 101 is movably housed in circular recess 126 on port unit 111 preventing these units from disconnection and serving as a guide for the rotation of port unit 111. In obtained blood stream channel including passageways 115, 112, 113, the minimal area of passage is substantially more than one of the indication channel, which is internal channel 110 of needle 105. This allows substantial increasing in the blood rate and reducing in the duration of hemodialysis or blood collection procedure. This blood stream channel does not have abrupt changes of diameter and outstanding sharp elements, providing laminar blood flow without blood cell damages and blood clot formations. In the blood stream position, catheter unit 101 occludes the distal outlet of needle guard-needle port 114 preventing needle distal sharp end 106 from going out of the needle guard in a distal direction and fulfilling the role of the locking means.

After the accomplishment of hemodialysis or blood collection procedure, the user rotates port unit 111 through 900 relative to catheter unit 101 changing over the device into the finish position (not shown), wherein blood stream port 112 is disconnected from tubular catheter 102 and both ports 112 and 114 are occluded from the distal side by catheter unit 101. This position prevents a blood leakage from blood stream port 112 after the withdrawal of tubular catheter 102 from the patient's vein and provides entire protection of needle distal sharp end 106 from any contact with personnel. The device has the fixing means in the form of elastic latch 127 located on catheter unit 101 and interacting with notches 128, 129, 130, located on rotatable port unit 111 for preventing spontaneous disturbance of the device positions. In the penetration and needle retraction positions, latch 127 engages notch 129, in the blood stream position, it engages notch 128, and in the finish position, it engages notch 130. Latch 127, notches 128-130, and finned surfaces 123, 124 are the controlling means for the change over of the device positions. The device is provided with the unit's sealing means in the form of elastic members 132 located between catheter unit 101 and port unit 111 and preventing the blood leakage from the zone of movable connection of these units. Sealing members 122, 132, as well as the above mentioned occlusion of blood stream port 112 and needle port 114 in the finish position reliably prevent the device from blood leakages throughout all stages of device operation.

In version embodiment (not shown), the device change over into the blood stream position is implemented by the rotation of the port unit relative to the catheter unit around the axis, which is perpendicular to the axis of the tubular catheter, thereby communicating the internal channel of the tubular catheter and the blood stream port and forming the blood stream channel.

In version embodiment (not shown), the needle retraction means is automatically operating and includes a compression spring mounted between the distal end of needle hub 107 and the proximal end of needle port 114, as well as a trigger member holding said spring in the stressed state in the penetration position and releasing the spring under user's action to retract the needle by this spring to the needle retraction position.

In version embodiment (not shown), the needle unit has detachable connection with the port unit and is removed from the device after the insertion of said tubular catheter into patient's vein. Moreover, the needle port sealing means includes an occluding sealing means preventing the device from the blood leakage after the needle removal from the device.

In version embodiments (not shown), the indication channel is formed with the needle internal channel connecting with the blood stream port via a needle lateral opening and an additional channel, and the blood stream port or the tube connecting the blood stream port with the external device serves as the indication cavity.

These not shown version embodiments correspond to the scope and spirit of the invention and its claims and differ from shown versions only by their embodiments accessible for usual engineers skilled in the art.

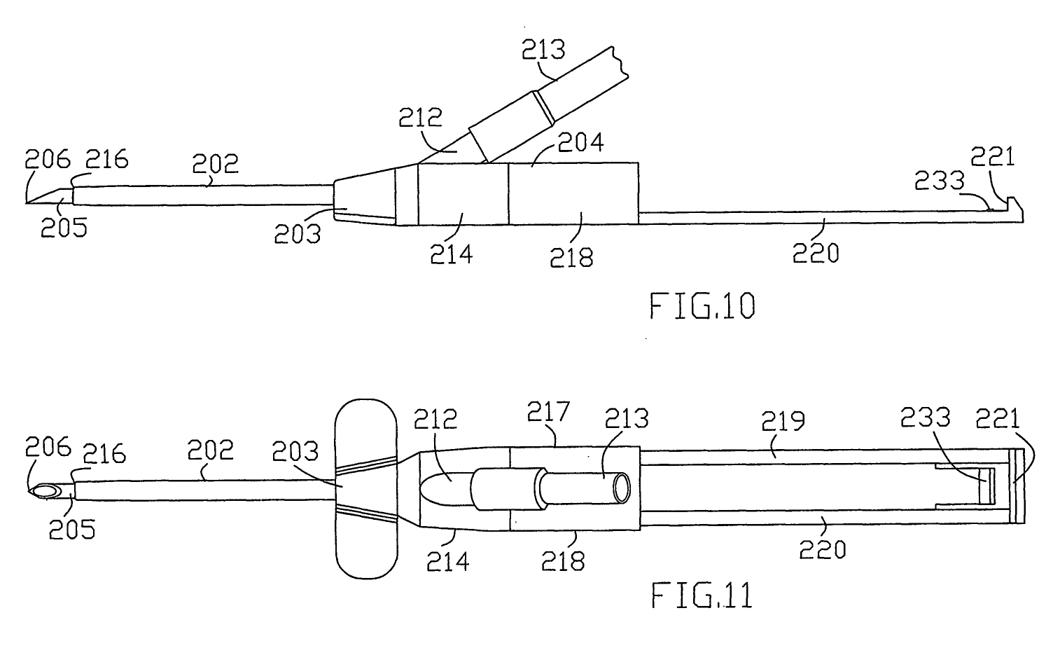

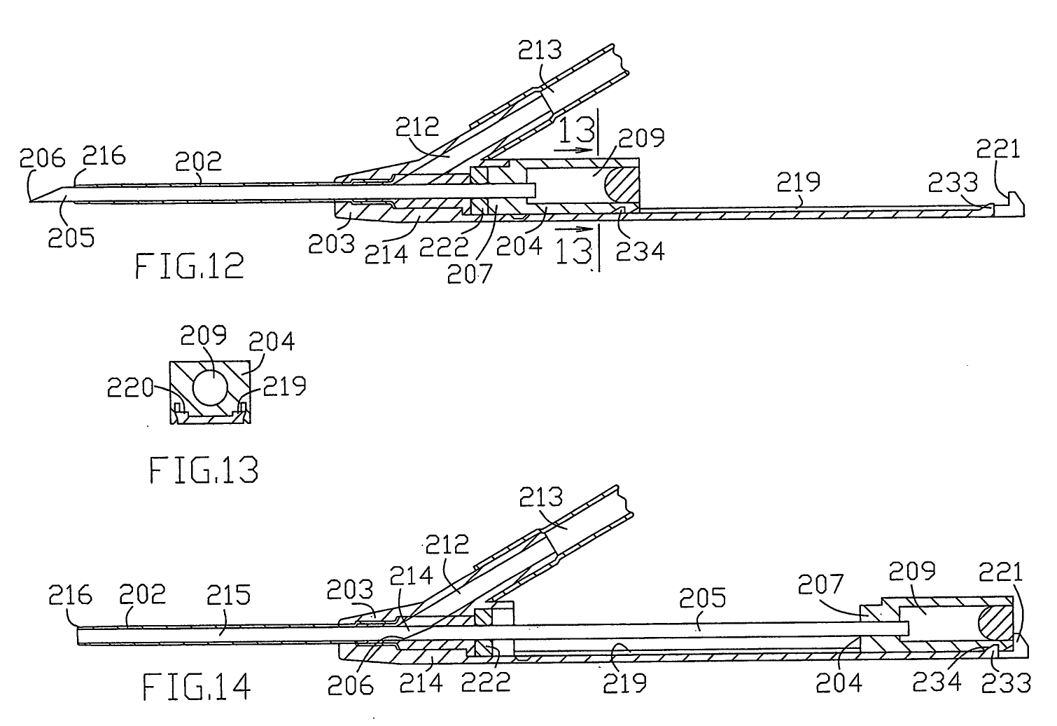

The device version, shown in FIGS. 10 to 14, has many elements identical to those of the previous version and having the same last two designation numerals. The description of these elements in the previous version mainly relates also to the present version. At the same time, the present version has certain distinctions, which are considered in the following.

In the device, shown in FIGS. 10 to 14, catheter hub 203, blood stream port 212, and needle port 214 are incorporated in an integral unit. The change over of this device from the insertion position (FIG. 12) into the needle retraction position is simultaneously the change over into the blood stream position (FIG. 14), as these two last positions are coincident. The user retracts needle 205 manually by means of pulling members 217, 218, which are retraction and controlling means. Guides 219, 220 serve for the guidance and support of needle hub 207 during the retraction. In the needle retraction—blood stream position, needle 205 is removed from tubular catheter 202, distal sharp end 206 is located inside needle port—needle guard 214 and locked in this position with stopping member 221 and the locking means including latch member 233 elastically connected with guides 219, 220 and notch 234 on needle unit 204. In the blood stream position, the blood stream channel, including passageways 215, 212, 213, has the minimal area of passage substantially more than one of the indication channel (needle internal channel). This allows increasing in blood rate and decreasing in medical procedure duration. The blood stream channel is located aside needle 205 and has relatively smooth transition zones between the passageways, thereby preventing the blood impairment.

1. A safety catheter device, comprising:
a tubular catheter having a catheter hub at its proximal end and designed for inserting into patient' vein and blood supply to the external device such as a hemodialysis or blood collection set;
a blood stream port designed for the connection of said tubular catheter with said external device;
a penetrating means designed for the introduction of said tubular catheter into patient's vein and comprising a needle with a distal sharp end, a needle hub, and a needle port located adjacent to said tubular catheter and serving as a passageway for introducing said needle into said tubular catheter;
an indication means designed for the indication of getting said needle distal sharp end into patient's vein and having an indication cavity with transparent external walls and an indication channel designed for blood supply into said indication cavity and including a penetrating means channel participating in the blood supply into said indication cavity;
a blood stream channel formed with an internal channel of said tubular catheter and said blood stream port and having a minimal area of passage, which is more than a minimal area of passage of said indication channel;
mutual dispositions of device elements are determined with a penetration position, a needle retraction position, and a blood stream position, wherein:
in said penetration position, said needle is passing through said needle port and said internal channel of tubular catheter, said needle distal sharp end is extended distally of the distal end of said tubular catheter, and said indication channel is open for blood supply into said indication cavity;
in said needle retraction position, followed by said penetration position, said needle is removed from said tubular catheter to provide the possibility of complete opening of said blood stream channel;
in said blood stream position, said blood stream channel is completely open and ready for blood supply from patient's vein into said external device;
a needle retraction means for the retraction of said needle from said penetration position to said needle retraction position;
a controlling means for the change-over of said device positions;
a needle port sealing means preventing the blood leakage through said needle port outside of the limits of said needle port;
a protector means including a guard, which protects said needle distal sharp end from contact with personnel after said needle retraction.

2. The safety catheter device of claim 1, wherein there is a catheter unit including said tubular catheter and said catheter hub, as well as a port unit including said needle port and said blood stream port, and said port unit is movably connected to said catheter unit.

3. The safety catheter device of claim 2, wherein there is a unit's sealing means located between said catheter unit and said port unit to prevent a blood leakage from the zone of the connection of said units.

4. The safety catheter device of claim 2, wherein there is a mounting means preventing said catheter unit and port unit from spontaneous disconnection and, at the same time maintaining their mutual mobility.

5. The safety catheter device of claim 1, wherein said retraction means fulfils the function of said controlling means, as the needle retraction to said needle retraction position completely opens said blood stream channel, thereby changing over the device to said blood stream position.

6. The safety catheter device of claim 5, wherein said catheter hub, said needle port, and said blood stream port are incorporated in an integral unit.

7. The safety catheter device of claim 4, wherein said controlling means provides said change-over of said device positions by the displacement of said port unit relative to said catheter unit.

8. The safety catheter device of claim 7, wherein there is a fixing means for the fixation of said port unit relative to said catheter unit in said device positions so that said fixing means eliminates the spontaneous disturbance of said positions.

9. The safety catheter device of claim 7, wherein said displacement of port unit relative to catheter unit presents the rotation of said port unit around the axis, which is parallel to the axis of said tubular catheter.

10. The safety catheter device of claim 7, wherein said displacement of port unit relative to catheter unit presents the rotation of said port unit around the axis, which is perpendicular to the axis of said tubular catheter.

11. The safety catheter device of claim 7, wherein said displacement of port unit relative to catheter unit presents a linear movement.

12. The safety catheter device of claim 1, wherein said needle retraction means is manually operated and includes a needle pulling means in the form of external members connected with said needle hub, accessible and adapted to the operation contact with user's fingers, and allowing for the retraction of said needle from said penetration position to said retraction position.

13. The safety catheter device of claim 1, wherein said needle retraction means is automatically operating and includes a spring mounted between said needle hub and members connected with said needle port, as well as a trigger member holding said spring in the stressed state in said penetration position and releasing said spring under user's action to retract said needle by said spring to said needle retraction position.

14. The safety catheter device of claim 1, wherein said blood stream channel has a minimal area of passage, which is more than a minimal area of the internal channel of said needle.

15. The safety catheter device of claim 1, wherein said needle port fulfills a role of said guard.

16. The safety catheter device of claim 1, wherein there is a guide means for the guidance and support of said needle during its retraction to said needle retraction position.

17. The safety catheter device of claim 16, wherein said guide means presents the guides of said needle hub extended proximally of said needle port.

18. The safety catheter device of claim 1, wherein there are a stopping means preventing said needle distal sharp end from going out of said guard in a proximal direction during needle retraction into said needle retraction position, as well as a locking means preventing said needle distal sharp end from going out of said guard in a distal direction after needle retraction into said needle retraction position.

19. The safety catheter device of claim 18, wherein said locking means includes an elastic latch member and a recess one of which is located on the element immovably connected with said needle and the other on the element immovably connected with said needle port, so that in said needle retracted position said latch member and recess engage, preventing said needle distal sharp end from going out of said guard in a distal direction.

20. The safety catheter device of claim 2, wherein said locking means presents a portion of said catheter unit, which occludes the distal outlet of said guard as a result of the displacement of said port unit relative to said catheter unit, thereby eliminating the possibility of the movement of said needle distal sharp end in a distal direction.

21. The safety catheter device of claim 1, wherein said needle remains in the device in said blood stream position.

22. The safety catheter device of claim 1, wherein said needle in the protected state and said needle guard are removed from the device after the insertion of said tubular catheter into the patient's vein.

23. The safety catheter device of claim 22, wherein said needle port sealing means includes an occluding sealing means preventing the blood leakage from the device after said needle removal.

24. The safety catheter device of claim 1, wherein said indication cavity presents a flash chamber attached to said needle hub on its proximal side, and said indication channel presents an internal channel of said needle.

25. The safety catheter device of claim 1, wherein said indication means includes said blood stream port, and said indication channel includes a needle internal channel connected with said blood stream port via a needle lateral opening and an additional channel.

26. The safety catheter device of claim 25, wherein said blood stream port also fulfils the role of said indication cavity.

27. The safety catheter device of claim 25, wherein said indication cavity presents a portion of a tube communicating said blood stream port with said external device.

28. The safety catheter device of claim 7, wherein there is also a finish position, which is set after said blood stream position, and in said finish position said blood stream port is disconnected from said tubular catheter and sealed to prevent a blood leakage from said blood stream port after the withdrawal of said tubular catheter from patient's vein, said needle port is disconnected from said tubular catheter, and said needle distal sharp end is protected from any contact with personnel, therewith, the change-over of the device into said finish position is implemented with said controlling means.

29. A safety catheter device, comprising:
a catheter unit including a tubular catheter and a catheter hub disposed at proximal end of said tubular catheter, and said tubular catheter is designed for inserting into patient' vein and blood supply to the external device such as a hemodialysis or blood collection set;
a blood stream port designed for the connection of said tubular catheter with said external device;
a penetrating means designed for the introduction of said tubular catheter into patient's vein and comprising a needle with a distal sharp end, a needle hub, and a needle port located adjacent to said tubular catheter and serving as a passageway for introducing said needle into said tubular catheter;
a port unit including said blood stream port and said needle port and movably connected to said catheter unit;
an indication means designed for the indication of getting said needle distal sharp end into patient's vein and having an indication cavity with transparent external walls and an indication channel designed for blood supply into said indication cavity and including a penetrating means channel participating in the blood supply into said indication cavity;
a blood stream channel formed with an internal channel of said tubular catheter and said blood stream port and having a minimal area of passage, which is more than minimal area of passage of said indication channel;
mutual dispositions of device elements are determined with a penetration position, a needle retraction position, and a blood stream position, wherein:
in said penetration position, said needle is passing through said needle port and said internal channel of tubular catheter, said needle distal sharp end is extended distally of the distal end of said tubular catheter, and said indication channel is open for blood supply into said indication cavity;
in said needle retraction position, followed by said penetration position, said needle is removed from said tubular catheter to provide the possibility of complete opening of said blood stream channel;
in said blood stream position, said blood stream channel is completely open and ready for blood supply from patient's vein into said external device;
a needle retraction means for the retraction of said needle from said penetration position to said needle retraction position;
a controlling means for the change-over of said device positions by the displacement of said port unit relative to said catheter unit;
a needle port sealing means preventing the blood leakage through said needle port outside of the limits of said needle port;
a protector means including a guard, which protects said needle distal sharp end from contact with personnel after said needle retraction.

30. A safety catheter device, comprising:
a tubular catheter having a catheter hub at its proximal end and designed for inserting into patient' vein and blood supply to the external device such as a hemodialysis or blood collection set;
a blood stream port designed for the connection of said tubular catheter with said external device;
a penetrating means designed for the introduction of said tubular catheter into patient's vein and comprising a needle with a distal sharp end, a needle hub, and a needle port located adjacent to said tubular catheter and serving as a passageway for introducing said needle into said tubular catheter;
said catheter hub, said needle port, and said blood stream incorporated in an integral unit;
an indication means designed for the indication of getting said needle distal sharp end into patient's vein and having an indication cavity with transparent external walls and an indication channel designed for blood supply into said indication cavity and including a penetrating means channel participating in the blood supply into said indication cavity;
a blood stream channel formed with an internal channel of said tubular catheter and said blood stream port and having a minimal area of passage, which is more than minimal area of passage of said indication channel;
mutual dispositions of device elements are determined with a penetration position, and a needle retraction position, wherein:
in said penetration position, said needle is passing through said needle port and said internal channel of tubular catheter, said needle distal sharp end is extended distally of the distal end of said tubular catheter, and said indication channel is open for blood supply into said indication cavity;
in said needle retraction position, followed by said penetration position, said needle is removed from said tubular catheter, thereby providing the complete opening of said blood stream channel;
a needle retraction means for the retraction of said needle from said penetration position to said needle retraction position;
a needle port sealing means preventing the blood leakage through said needle port outside of the limits of said needle port;
a protector means including a guard, which protects said needle distal sharp end from contact with personnel after said needle retraction.

31. The safety catheter device of claim 18, wherein said locking means presents a portion of said catheter unit, which occludes the distal outlet of said guard as a result of the displacement of said port unit relative to said catheter unit, thereby eliminating the possibility of the movement of said needle distal sharp end in a distal direction.
